(19) United States
(12) Patent Application Publication
(10) Pub. No.: US 2018/0291336 A1
(43) Pub. Date: Oct. 11, 2018

(54) METHOD OF MANUFACTURING AND PURIFYING EXOSOMES FROM NON-TERMINALLY DIFFERENTIATED CELLS

(71) Applicant: Robert John Petcavich, The Woodlands, TX (US)

(72) Inventor: Robert John Petcavich, The Woodlands, TX (US)

(21) Appl. No.: 15/947,329

(22) Filed: Apr. 6, 2018

Related U.S. Application Data
(60) Provisional application No. 62/483,059, filed on Apr. 7, 2017.

(51) Int. Cl. C12N 5/071; B01D 17/02; B01D 43/00

(52) U.S. Cl. CPC C12N 5/0602; B01D 43/00; B01D 17/0217

(57) ABSTRACT

The present invention discloses a high volume manufacturing process for the production and purification of exosomes. In one embodiment, a hollow fiber perfusion reactor is used to expand stem cells. The growth media used from the stem cell expansion is captured, filtered, centrifuged and exosomes isolated from the waste effluent. In one embodiment, stem cells are derived from bone marrow, fat, blood, cord blood and induced pluripotent stem cells are expanded and the exosomes captured from the growth waste effluent. The exosomes can be used as therapeutics and diagnostics for a number of regenerative and chronic diseases.

### CROSS-REFERENCE TO RELATED APPLICATIONS

This application claims the benefit of the filing date of U.S. application Ser. No. 62/483,059, filed on Apr. 7, 2017, the disclosure of which is incorporated by reference herein.

### FIELD OF THE INVENTION

The present subject matter relates to systems and methods for large-scale continuous production and purification of exosomes that are generated during non-terminally differentiated cell expansion and harvesting in a hollow fiber perfusion bioreactor.

### BACKGROUND

Exosomes are cell-derived vesicles that are present in many and perhaps all eukaryotic fluids, including blood, urine, and cultured medium of cell cultures. The reported diameter of exosomes is between 30 and 100 nm, which is larger than low-density lipoproteins (LDL) but much smaller than, for example, red blood cells. Exosomes are either released from the cell when multivesicular bodies fuse with the plasma membrane or released directly from the plasma membrane. Evidence is accumulating that exosomes have specialized functions and play a key role in processes such as coagulation, intercellular signaling, and waste management. Consequently, there is a growing interest in the clinical applications of exosomes. Exosomes can potentially be used for prognosis, for therapy, and as biomarkers for health and disease.

Exosomes contain various molecular constituents of their cell of origin, including proteins and RNA. Although the exosomal protein composition varies with the cell and tissue of origin, most exosomes contain an evolutionarily conserved common set of protein molecules. The protein content of a single exosome, given certain assumptions of protein size and configuration, and packing parameters, can be about 20,000 molecules. The cargo of mRNA and miRNA in exosomes was first discovered at the University of Gothenburg in Sweden. In that study, the differences in cellular and exosomal mRNA and miRNA content was described, as well as the functionality of the exosomal mRNA cargo. Exosomes have also been shown to carry double-stranded DNA.

Exosomes can transfer molecules from one cell to another via membrane vesicle trafficking, thereby influencing the immune system, such as dendritic cells and B cells, and may play a functional role in mediating adaptive immune responses to pathogens and tumors. Therefore, scientists that are actively researching the role that exosomes may play in cell-to-cell signaling, often hypothesize that delivery of their cargo RNA molecules can explain biological effects. For example, mRNA in exosomes has been suggested to affect protein production in the recipient cell. However, another study has suggested that miRNAs in exosomes secreted by mesenchymal stem cells (MSC) are predominantly pre- and not mature miRNAs. Because the authors of this study did not find RNA-induced silencing complex-associated proteins in these exosomes, they suggested that only the pre-miRNAs but not the mature miRNAs in MSC exosomes have the potential to be biologically active in the recipient cells.

Conversely, exosome production and content may be influenced by molecular signals received by the cell of origin. As evidence for this hypothesis, tumor cells exposed to hypoxia secrete exosomes with enhanced angiogenic and metastatic potential, suggesting that tumor cells adapt to a hypoxic microenvironment by secreting exosomes to stimulate angiogenesis or facilitate metastasis to more favorable environment.

Increasingly, exosomes are being recognized as potential therapeutics as they have the ability to elicit potent cellular responses in vitro and in vivo. Exosomes mediate regenerative outcomes in injury and disease that recapitulate observed bioactivity of stem cell populations. Mesenchymal stem cell exosomes were found to activate several signaling pathways important in wound healing (Akt, ERK, and STAT3) and bone fracture repair. They induce the expression of a number of growth factors (hepatocyte growth factor (HGF), insulin-like growth factor-1 (IGF1), nerve growth factor (NGF), and stromal-derived growth factor-1 (SDF1)). Exosomes secreted by human circulating fibrocytes, a population of mesenchymal progenitors involved in normal wound healing via paracrine signaling, exhibited in-vitro proangiogenic properties, activated diabetic dermal fibroblasts, induced the migration and proliferation of diabetic keratinocytes, and accelerated wound closure in diabetic mice in vivo. Important components of the exosomal cargo were heat shock protein-90α, total and activated signal transducer and activator of transcription 3, proangiogenic (miR-126, miR-130a, miR-132) and anti-inflammatory (miR124a, miR-125b) microRNAs, and a microRNA regulating collagen deposition (miR-21). Exosomes can be considered a promising carrier for effective delivery of small interfering RNA due to their existence in body's endogenous system and high tolerance. Patient-derived exosomes have been employed as a novel cancer immunotherapy in several clinical trials.

Exosomes offer distinct advantages that uniquely position them as highly effective drug carriers. Composed of cellular membranes with multiple adhesive proteins on their surface, exosomes are known to specialize in cell-cell communications and provide an exclusive approach for the delivery of various therapeutic agents to target cells. For example, researchers used exosomes as a vehicle for the delivery of the cancer drug Paclitaxel. They placed the drug inside exosomes derived from white blood cells, which were then injected into mice with drug-resistant lung cancer. Importantly, incorporation of Paclitaxel into exosomes increased cytotoxicity more than 50 times as a result of nearly complete co-localization of airway-delivered exosomes with lung cancer cells.

Exosomes contribute to organ development and mediate regenerative outcomes in injury and disease that recapitulate observed bioactivity of stem cell populations. Encapsulation of the active biological ingredients of regeneration within non-living exosome carriers may offer process, manufacturing and regulatory advantages over stem cell-based therapies.

Exosomes participate in key mechanistic pathways in development, organogenesis, wound healing and regeneration in adults by mediating inter-cell communication of key developmental morphogens and other signaling elements. It would therefore be desirable to have a large-scale cost effective process to manufacture large quantities of stem cell derived exosomes for therapeutic and diagnostic purposes. The present invention provides, among other things, a cost effective method of producing large quantities and subsequently purifying exosomes from stem cells for therapeutic and diagnostic applications.

### SUMMARY

Various embodiments of the present subject matter provide an automated manufacturing platform for the large-scale production and purification of exosomes from non-terminally differentiated cells using a hollow fiber perfusion bioreactor.

Various embodiments of the present subject matter provide a large scale-manufacturing platform for producing and purifying exosomes derived from, for example, embryonic, mesenchymal, hematopoietic, induced pluripotent, primary and cancer non-terminally differentiated cells.

Various embodiments of the present subject matter separate and purify the exosomes generated from growth media effluent from a large scale-manufacturing hollow fiber perfusion reactor platform for therapeutic and diagnostic use.

Other advantages of the present subject matter will become apparent to persons of skill in the art upon reading and understanding the following detailed description. This Summary is an overview of some of the teachings of the present application and not intended to be an exclusive or exhaustive treatment of the present subject matter. Further details about the present subject matter are found in the detailed description and appended claims. The scope of the present invention is defined by the appended claims and their legal equivalents.

### BRIEF DESCRIPTION OF THE DRAWINGS

FIG. 1 shows a cross section of a typical hollow fiber reactor.

FIG. 2 shows an image of a typical hollow fiber reactor fluid loop used in a hollow fiber perfusion bioreactor (HFPB).

FIG. 3 shows a flow chart of an exemplary manufacturing process, e.g., for iPSc.

FIG. 4 shows processes for exosome isolation that include ultracentrifugation or density gradient ultracentrifugation.

FIG. 5 is a flow chart for an exemplary exosome isolation process.

FIG. 6 is a schematic of an exemplary exosome isolation process.

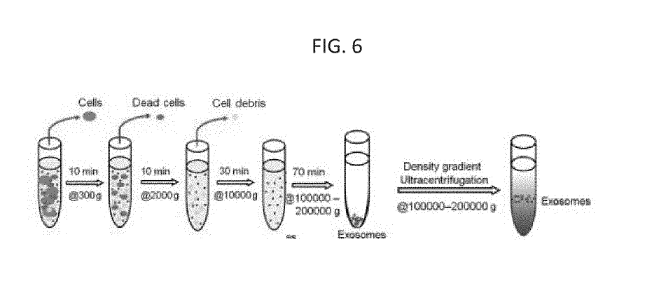

### DETAILED DESCRIPTION

The following discussion provides various exemplary embodiments of the present subject matter. Although one or more of these embodiments may be preferred, the embodiments disclosed should not be interpreted, or otherwise used, as a limiting the scope of the disclosure, including the claims. One skilled in the art will understand that the following description has broad application, and the discussion of any embodiment is meant only to be exemplary of that embodiment, and not intended to intimate that the scope of the disclosure, including the claims, is limited to that embodiment.

This patent application incorporates by reference the subject matter in U.S. Provisional Patent Application Ser. No. 62/347,208, entitled: METHOD OF MANUFACTURING NEURON PROGENITOR CELLS DERIVED FROM HUMAN PLURIPOTENT STEM CELLS USING A HOLLOW FIBER BIOREACTOR, filed Jun. 8, 2016 (“the '208 Provisional). The '208 Provisional relates generally to the field of mammalian stem cells, e.g., human-derived induced pluripotent stem cells (iPSc), automated expansion using a hollow fiber bioreactor. The '208 Provisional disclosure describes, among other things, at least one way to manufacture neuron progenitors from iPScs on a large scale using an automated hollow fiber bioreactor.

### Exemplary Method of Manufacturing Cells on a Hollow Fiber Bioreactor

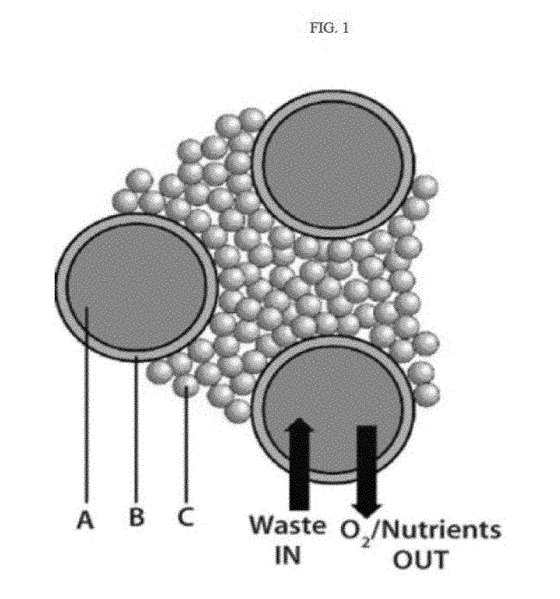

Hollow-fiber-based technologies in general are used in many applications, from tangential-flow filtration to prokaryotic biofilms in wastewater treatment. Here, hollow-fiber perfusion bioreactor (HFPB) technology is employed in the culture of mammalian cells. Mammalian cells can be generally seeded within a cartridge intra capillary space (IC), labeled A in FIG. 1, and outside the hollow fibers in what is referred to as the extra capillary space (EC), labeled B in FIG. 1. The cells are labeled C in de the hollow fibers in what is referred to as the extra capillary space (EC), labeled B in FIG. 1 and reside in the EC space. The IC and EC are thus separated by a fiber wall which acts as a membrane. Culture medium is pumped through the lumen of the hollow fibers, allowing nutrients and metabolic products to diffuse both ways across the fiber walls. Having passed through the cartridge, medium can be either oxygenated and returned to it or collected while fresh medium is introduced.

In most culture systems, cells are seeded in a medium containing a great excess of nutrients and no metabolic products. They progress for a matter of days in an environment of declining nutrients and increasing products, only to be suddenly exposed to the original media composition again (when the culture is split into fresh media) or to a slightly different variation of the original formulation (during serial adaptation of a culture to a new medium). Recent advances in metabolic flux analysis support the significance of such exposure to a variable and even discontinuous cycle of nutrient and metabolic products. However, the relative constancy of the culture matrix and ambient chemical environment in an HFPB system provides a more consistent and physiologic culture environment. Because freshly oxygenated medium is continually exchanged through an immobilized culture, cells exist in an environment of relatively constant metabolite and growth factor concentration. This benefits a number of applications. For example, some primary and specialized cell lines tend to regulate critical pathways, differentiate, or “shock” in response to significant or sudden changes in their medium.

The HFPB culture chamber environment is continuously controllable in real time. Because HFPB systems possess such an efficient medium-exchange mechanism, it is easy to alter the input medium composition (and therefore the ambient cellular environment) whenever desired. This differs from fed-batch cultures, which allow only the addition of a bolus of nutrients or reagents on top of existing media components. Furthermore, high-density culture in the controlled hydrodynamic conditions of an HFPB can provide a microenvironment of directional flow, establishing a gentle interstitial gradient within the cell mass for autocrine stimulation, cell alignment, and desirable cell-cell or cell-surface interactions.

Because an HFPB cell culture (on the EC side of the fibers) exists at concentrations ≥100× that of standard suspension cultures, less serum is needed and product may be harvested at many times the concentration of that from most other systems. Other benefits include facilitation of adapting cultures to serum-free media and better support for conditioned media and autocrine-dependent cultures. The initial volume of a circulating loop can be very low when a culture is seeded, then raised as the cell number increases, thereby maintaining a more constant cell/medium ratio.

Cells in HFPB culture are separated from the bulk of their medium by a membrane (e.g., fiber wall) of definable composition and porosity. The cells essentially experience two different volumes: That seen for low-molecular-weight components such as glucose and lactate is relatively large, whereas the volume seen for larger components and some stimulatory cytokines is ≥100× smaller. Because both the culture (EC) and perfused medium (fiber lumen; IC) sides of the system are accessible to sampling and feeding, it is common to maintain and monitor particular components within each distinct space. This provides for many valuable functions in operation.

For example, the volume of the cell-containing compartment can be quite small, so products can be harvested at 100× or higher concentration than from suspension culture. By matching a reactor's fiber porosity to cell characteristics, products may be accumulated, maintained, and measured on either side of the system. In one application, macromolecular culture factors can be introduced or allowed to accumulate within the EC side.

Combining such features as an unlimited nutrient supply and the ability to “debulk” a culture through the cartridge ports allows an HFPB system to be maintained at relative equilibrium for several months or longer. Continuous production over long periods provides several benefits over batch cultures: consistency in culture condition, dramatically increased production per unit footprint and culture volume, continuous or daily product harvest that allows timely stabilizing treatment or storage conditions, and the option of continually removing products from a culture that might be toxic or inhibitory to cells.

The large number of ex vivo expanded cells that are required in many high throughput drug screenings, roughly 2 million per 384 plate, and clinical cell therapy protocols (>200 million per patient) make standard culture conditions problematic and expensive, resulting in the need for extensive personnel and facilities resources, and the potential for contamination. To meet such clinical demand, a robust automated and closed cell expansion method is optimal. The HFPB system useful in the present methods is a functionally closed, automated hollow fiber bioreactor system designed to reproducibly grow both adherent and suspension cells in either GMP or research laboratory environments. The system has been used for the ex vivo expansion of clinical-scale quantities of human mesenchymal stem cells (MSC). MSCs from precultured cells were expanded in the system with media consisting of α-MEM, 10 percent FBS, 1× GlutaMAX and no additional antibiotics/antimycotics or supplementary factors. Glucose and lactate levels in the media were monitored to maintain optimal culture conditions. The HFPB system-expanded MSCs met all typical MSC characteristics for phenotype and differentiation. Cell numbers suitable for therapeutic dosages of MSC were generated in five days from initial cell loads of about 15 to about 20 million cells.

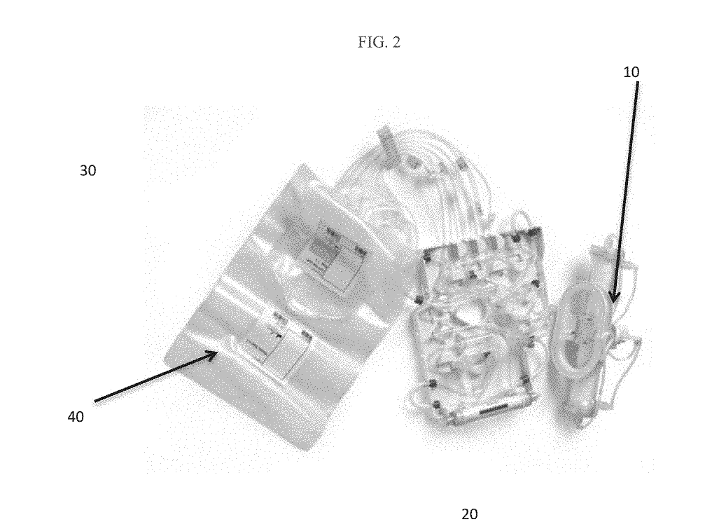

As shown in de the hollow fibers in what is referred to as the extra capillary space (EC), labeled B in FIG. 2, the bioreactor culture system is comprised of a synthetic hollow fiber bioreactor 10 that is part of a sterile closed-loop circuit for media and gas exchange, a gas exchange module 20, a pre-attached waste bag 30 and a pre-attached cell harvest bag 40.

A wide range of materials, such as polysulfone and cellulose derivatives, may be used for the hollow fibers. Molecular weight cutoffs begin at 5 kDa and go up to virtually any desired upper limit. The fiber materials can vary in such properties as percent porosity, molecular weight cut-off, and hydrophilicity, and they can be further modified during either manufacturing or their actual application to introduce defined functionalities onto their surfaces. Characteristics of an HFPB system are: Extremely high binding culture surface to volume ratios, immobilization of cells at a very high (biomimetic) density on a porous matrix supporting prolonged culture selectable porosity of the fibers for such purposes as concentration of secreted products or delivered media, differentiation factors, and enzymes of interest.

In one embodiment, the bioreactor and fluid circuit are a single-use disposable set that is mounted onto the Quantum system unit that can be purchased from TERUMOBCT Corporation in Lakewood, Colo. In one embodiment, the bioreactor may be comprised of about 11,500 hollow fibers with a total intracapillary (IC) surface area of 2.1 m². Typical culture manipulations (e.g., cell seeding, media exchanges, differentiation factors, trypsinization, cell harvest, etc.) are managed by the computer-controlled system using pumps and automated valves, which direct fluid through the disposable set and exchanges gas with the media. The functionally closed nature of the disposable set is maintained through the sterile docking of bags used for all fluids; these bag connections/disconnections all utilize sterile connection technology. In one embodiment, gas control in the system is managed using a hollow fiber oxygenator (gas transfer module 20, de the hollow fibers in what is referred to as the extra capillary space (EC), labeled B in FIG. 2). In one embodiment, gas is supplied from a user-provided premixed gas tank. By choosing a tank with the desired gas mixture, the user can expand cells at their optimal gas concentration. In one embodiment, the IC membrane of the bioreactor may be coated with an adhesion promoter such as fibronectin, matrigel, gelatin or combinations of the aforementioned promoters to allow the attachment of adherent cell populations, such as iPSc, neuron progenitor cells or cardiomyocytes.

In one embodiment, after loading a new cell expansion set using the Load Cell Expansion Set Task (every run/passage requires the use of a new disposable set to assure process sterility), the disposable set may be primed with PBS in the 4 L Media Bag that has been connected to the Cell Inlet line of the disposable set using a Terumo® TSCD or TSCDII sterile connection device. In one embodiment, the Media Bag Accessory is filled with fluid from reagent bottles under a biosafety cabinet utilizing a tubing pump (Cole-Parmer Masterflex® L/S Tubing Pump with the Easy-Load II pump header) to pump the fluid into the Media Bag Accessory; all sealing of disposable tubing lines is done with an RF Sealer (Sebra Omni™ 2600 Sealer). In one embodiment, once the disposable set has been primed, the bioreactor is coated overnight with 10 mg of matrigel to promote cell adhesion using the Coat Bioreactor Task. In one embodiment, the matrigel is prepared by solubilizing 10 mg of matrigel in 20 mL of sterile H₂O for 30 minutes, bringing the solution volume to 100 mL with 80 mL of PBS, transferring the matrigel solution to a Cell Inlet Bag in a biosafety cabinet. In one embodiment, after the overnight bioreactor coating, any excess matrigel is washed from the bioreactor set and the cell culture media is introduced into the set utilizing the IC/EC Washout Task allowing the exchange of PBS solution with Media, which has been filled into a 4 L Media Bag accessory and sterile connected to the IC Inlet Line. To assure that the newly introduced Media is properly oxygenated, in one embodiment, the Condition Media Task is run for a minimum of 10 minutes. In one embodiment, the gas mixture used is 20 percent O₂, 5 percent CO₂ and the balance N₂. At this point the disposable set is ready to be used for cell loading and expansion.

In one embodiment, eighty to one hundred million NPCs are transferred into the cell inlet bag of the HFPB and the volume is brought up to 100 mL with the growth media of interest. In one embodiment, the bag is then connected to the inlet line of the HFPB and the cells loaded onto the IC side of the bioreactor utilizing the Load Cells with circulation program. This step is designed to allow uniform distribution of the cells throughout the IC side of the bioreactor. Once this task is complete, in one embodiment, the system is put in the Attach cells task mode, which allows the cells to adhere to the coated IC membrane surface. In one embodiment, the IC media flow rate is zero to allow the cell attachment, while the EC flow rate may be approximately 30 mL/min to maintain oxygenation in the bioreactor. In one embodiment, the cells are allowed to attach for 24 to 48 hours optionally followed by a rapid IC washout to remove any nonadherent cells.

In one embodiment, cells are grown for at least three to five days utilizing the Feed Cells Task with fresh media added to the IC side of the bioreactor and the IC inlet rate adjusted as required by the rate of glucose consumption and lactate generation in the system which is monitored from a sample port twice daily. The IC waste valve is open for the duration of the expansion phase to allow waste media to collect in the waste bag to prevent protein accumulation in the IC loop.

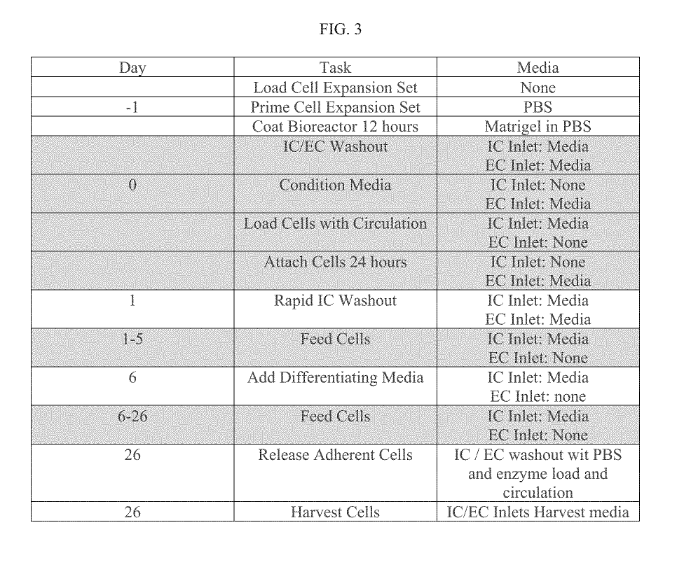

In one embodiment, after 5-7 days, the expansion is complete. In one embodiment, at this point of the expansion there are 500 million to 2 billion cells in the HFPB. In one embodiment, cells are released from the IC membrane of the bioreactor using a 0.25 percent trypsin-EDTA or equivalent enzyme package as the adherent cell release agent. Other enzymes can be used depending on the type of hollow fibers used in the bioreactor such as cellulase and collagenase. In one embodiment, the Release Adherent Cells Task is used to flush media from the system with PBS, then to fill the bioreactor with the enzyme solution that is circulated in the bioreactor from 4 to 10 minutes. Once this task is complete, released cells are harvested utilizing the Harvest task with the proper harvest media in the Harvest bag attached to the IC inlet line. Once the harvest is complete the harvest bag is sealed and detached from the reactor. The cells can be subsequently removed from the bag for cell counting, viability analysis, phenotyping, morphology, and differentiation assays. FIG. 3 is an exemplary flow chart for culturing cells in a HFBR.

An example of materials and conditions for neural progenitor cells is provided below.

A. Neural Progenitor Cells Materials/Equipment
a. Consumables
   i. Quantum Expansion Set (1×)
   ii. 4 L Media Bag (9×)
   iii. Cell Inlet Bag (5×)
   iv. Waste Bag (5×)
   v. Gas Supply (5% CO₂, 20% O₂, Balance with N₂)
   vi. Glucose strips
   vii. Lactate strips
b. Media/Reagents
   i. DMEM/F12 (Cat#: 11330-032, Corning)
   ii. PBS without calcium and magnesium
   iii. Poly-L-Ornithine (PLO; Cat#: P3655, Sigma)
   iv. Laminin (Cat#: 23017-015, Invitrogen)
   v. Accutase Cell Dissociation Reagent (Cat#: A11105-01, Thermo)
   vi. B27, N2 Supplements
   vii. Fibroblast Growth Factor-2 (FGF-2)
   viii. BDNF (Brain-derived neurotropic factor)
   ix. GDNF (Glial cell-derived neurotropic factor)

B. Procedures (Expansion)
a. Load Cell Expansion set
b. Prime the expansion set with 2 Ls of DMEM/F12 in a “Cell” Line
c. Prepare extracellular matrix for neural progenitor cells with PLO/Laminin.
   i. PLO preparation: Dissolve the PLO powder in cell culture water to make a stock solution of 10 mg/mL and aliquot 1 mL portions. Dilute the stock solution of 10 mg/mL in cell culture water to yield 10 μg/mL (1:1000).
      1. Put 100 mL of cell culture water in an empty 125 mL bottle, and add 100 μL of the stock solution (10 mg/mL) of PLO. Put the 100 mL solution in the reagent bag.
   ii. Laminin preparation: Dilute Laminin in PBS− (Sigma-Aldrich PS244) to yield 5 μg/mL (1:200)
      1. Put 100 mL of PBS− in an empty 125 mL bottle, and add 500 uL of the stock solution of Laminin to yield 5 μg/mL (1:200). Put the 100 mL solution in the reagent bag and store the reagent bag in a 4'C refrigerator.
   iii. Prepare 4 L of cell culture water in a media bag.
d. Coating Bioreactor
   i. Connect the prepared PLO reagent bag to the “reagent” line. Coat the bioreactor overnight at 37'C.
   ii. Next day, wash the bioreactor three times with 1.4 L of cell culture water prepared in step B,iii by performing IC/EC (intracapillary/extracapillary space) washouts.
      1. Each washout removes about 90% of IC/EC volume.
   iii. Disconnect the PLO reagent bag, and connect the Laminin filled reagent bag to the “reagent” line, and coat the bioreactor for at least 2 hours.
e. Make 2 L of NBF media and introduce to a media bag.
   i. 0.5×N-2 supplement, 0.5×B-27 supplement and 1× Pencillin-Streptomycin in DMEM/F12 50/50 mix media.
f. Perform IC/EC washout with NBF and condition media until cells are ready to load.
g. Load about 80 million NPCs by dissociating about 10×10 cm dishes of NPCs.
h. Attach the cells
i. Feed the cells with appropriate volume of media and flow rate.
   i. After 24 hours of attachment iPSCs which is now day 1, start feeding the cells at 0.1 mL/minute. Measure the glucose/lactate and record every day until the expansion run is finished.
      1. On Day 2, make 300 ml of NBF and load it in a media bag. Feed the cells at 0.2 mL/minute.
      2. On Day 3, make 600 ml of NBF and load it in a media bag. Feed the cells at 0.4 mL/minute.
      3. On Day 4, make 1200 ml of NBF and load it in a media bag. Feed the cells at 0.8 mL/minute.
      4. On Day 5, make 2400 ml of NBF and load it in a media bag. Feed the cells at 1.6 mL/minute.
      5. **Media preparation for each day might be different and adjustable, but the final harvested number of cells would be about 1.3 billion cells, with possibly about 1.0 billion live cells.
j. Make a 2 L media bag with PBS− and a 100 ml reagent bag with Accutase (Keep the reagent bag in a 37'C water bath).
k. Start harvesting the expanded NPCs by performing IC/EC washout in the bioreactor once with the 2 L PBS−, and then dissociating with pre-heated 100 ml Accutase for approximately 5 minutes.
l. All the harvested cells are in the “harvest” bag. Transfer the bag into a biosafety cabinet hood. Cut the harvest bag and put the cells in 50 mL conical tubes. Centrifuge at 200×g for 5 minutes and re-suspend the pellet with 100 mL of fresh NBF.
   Following the aforementioned protocol resulted in the following neuroprogenitor cell expansion results after 7 days.

### Average Cell Counts

1. Total cells: 841M (loaded app. 100M)
2. Live cells: 644M

### 3. Viability: 76%

### Exemplary Methods

In one embodiment, a method of purifying exosomes from cells, e.g., non-terminally differentiated cells, which are cells that are optionally expanded in a hollow fiber perfusion reactor is provided. In one embodiment, the method includes providing a cellular effluent from non-terminally differentiated mammalian cells cultured in media; separating the cellular effluent into the mammalian cells and a first supernatant comprising exosomes; optionally separating the first supernatant into cellular debris and a second supernatant comprising exosomes; optionally separating the second supernatant, for example, using filtration into a filtrate comprising exosomes: optionally subjecting the filtrate to ultracentrifugation to pellet the exosomes; optionally resuspending the pelleted exosomes; subjecting the exosomes to centrifugation and/or density gradient ultracentrifugation to isolate exosomes; and collecting the isolated exosomes. In one embodiment, the effluent is a stem cell effluent. In one embodiment, the cellular effluent is separated by centrifugation, e.g., about 700 up to about 800, for instance up to about 750, ×g. In one embodiment, the first supernatant is separated by centrifugation, e.g., subjected to from about 1500 up to about 2500, for instance about 2000, ×g. In one embodiment, the second supernatant is further subjected to centrifugation before filtration, for example, subjected from about 9,000 to up to about 11,000, e.g., about 10,000, ×g before filtration. In one embodiment, the filtrate is subjected from about 95,000 to up to about 105,000, e.g., about 100,000, ×g. In one embodiment, the filter is an about a 0.15 to about 10.3, e.g., about a 0.2, micron filter. In one embodiment, the resuspended exosomes are subjected to up to about 100,000×g for about 18 hours. In one embodiment, the method further comprises subjecting the isolated exosomes to solvent exchange. In one embodiment, the effluent is obtained from a hollow fiber bioreactor comprising a plurality of fibers that is part of a sterile closed-loop circuit for media and gas exchange; a gas exchange module; a waste bag; and a cell harvest bag, wherein the fibers are coated with a glycoprotein and a molecule or mixture that promotes cell attachment. In one embodiment, the mammalian cells are human cells. In one embodiment, the cells are neural progenitor cells, induced pluripotent stem cells, mesenchymal stem cells, embryonic stem cells or hematopoietic stem cells.

In one embodiment, a method of purifying exosomes from mammalian cells is provided that includes providing an isolated supernatant comprising exosomes and lacking cells, which supernatant is obtained from cultured mammalian cells; filtering the supernatant to provide a filtrate comprising exosomes; concentrating the filtrate via ultracentrifugation to provide for isolated exosomes; subjecting the isolated exosomes to density gradient ultracentrifugation to obtain isopycnically isolated exosomes; and collecting the isolated exosomes. In one embodiment, the cultured cells are non-terminally differentiated cells. In one embodiment, the isolated supernatant is provided by separating a cellular effluent from cells and/or cell debris by centrifugation. In one embodiment, the centrifugation is about 700 to about 800×g. In one embodiment, the cellular effluent is separated from cells and/or cell debris by centrifugation from about 1500 to about 2500×g. In one embodiment, the filtrate is subjected to centrifugation up to about 100,000×g. In one embodiment, the filter is an about 0.15 to an about 0.3 micron filter. In one embodiment, the effluent is obtained from a hollow fiber bioreactor comprising a plurality of fibers that is part of a sterile closed-loop circuit for media and gas exchange; a gas exchange module; a waste bag; and a cell harvest bag, wherein the fibers are coated with a glycoprotein and a molecule or mixture that promotes cell attachment. In one embodiment, the mammalian cells are human cells. In one embodiment, the cells are neural progenitor cells, induced pluripotent stem cells, mesenchymal stem cells, embryonic stem cells or hematopoietic stem cells.

In one embodiment, a method of purifying exosomes from mammalian cells is provided including providing an isolated supernatant comprising exosomes and lacking cells, which supernatant is obtained from cultured mammalian cells; filtering the supernatant to provide a filtrate comprising exosomes; concentrating the filtrate via ultracentrifugation to provide for isolated exosomes or subjecting filtrate to density gradient ultracentrifugation to obtain isopycnically isolated exosomes; and collecting the isolated exosomes.

In one embodiment, the cultured cells are non-terminally differentiated cells. In one embodiment, the isolated supernatant is provided by separating a cellular effluent from cells and/or cell debris by centrifugation. In one embodiment, the centrifugation is about 700 to about 800×g. In one embodiment, the cellular effluent is separated from cells and/or cell debris by centrifugation from about 1500 to about 2500×g. In one embodiment, the filtrate is subjected to centrifugation up to about 100,000×g. In one embodiment, the filter is an about 0.15 to an about 0.3 micron filter. In one embodiment, the effluent is obtained from a hollow fiber bioreactor comprising a plurality of fibers that is part of a sterile closed-loop circuit for media and gas exchange; a gas exchange module; a waste bag; and a cell harvest bag, wherein the fibers are coated with a glycoprotein and a molecule or mixture that promotes cell attachment. In one embodiment, the mammalian cells are human cells. In one embodiment, the cells are neural progenitor cells, induced pluripotent stem cells, mesenchymal stem cells, embryonic stem cells or hematopoietic stem cells.

### Exemplary Applications of Effluent

In one embodiment, the instant disclosure applies growth media effluent used in the HFBR process and subsequently mining the media effluent for large volumes of exosomes produced by the billions of cells created during the hollow fiber expansion process.

In order to facilitate the application of these unique extracellular vesicles, it is important that exosomes are specifically isolated from a wide spectrum of cellular debris and interfering components. The techniques employed in the isolation of exosomes should exhibit high efficiency and are capable of isolating exosomes from various sample matrices. To examine the quality of isolated exosomes, several optical and non-optical techniques have been developed to gauge their size, size distribution, morphology, quantity, and biochemical composition, e.g., via laser light scattering techniques. With the fast advances in science and technology, many techniques have been developed for the isolation of exosomes in appreciable quantity and purity. Each technique exploits a particular trait of exosomes, such as their density, shape, size, and surface proteins to aid their isolation. Variants within each group also bring about a unique set of advantages and disadvantages to exosome isolation. Table 1 summarizes various techniques used in the prior art to isolate and purify exosomes.

TABLE I
Comparison of exosome isolation techniques

| Isolation | Isolation | | |
|---|---|---|---|
| Technique | Principle | Potential Advantage | Potential Disadvantage |
| Ultracentrifugation- | Density, size, | Reduced cost and | High equipment cost, |
| based | and shape based | contamination risks | cumbersome, long run time, |
| techniques | sequential | with separation | and labor intensive low |
| | separations of | reagents, Large | portability - not available at |
| | particulate | sample capacity and | point-of-care, high speed |
| | constituents and | yields large amounts | centrifugation may damage |
| | solutes | of exosomes. | exosomes thus impeding |
| | | | downstream analysis. |
| Size- | Exosome | Ultrafiltration: Fast, | Ultrafiltration: low equipment |
| based | isolation is | does not require | cost, moderate purity of |
| techniques | exclusively | special equipment, | isolated exosomes, shear stress |
| | based on the size | good portability, direct | induced deterioration, |
| | difference | RNA extraction | possibility of clogging and |
| | between | possible. | vesicle trapping, exosomes loss |
| | exosomes and | SEC: High-purity | due to attaching to the |
| | other particulate | exosomes, gravity | membranes. |
| | constituents | flow preserves the | SEC: Moderate equipment |
| | | integrity and | cost, requires dedicated |
| | | biological activity; | equipment, not trivial to scale |
| | | superior | up, long run time. |
| | | reproducibility, | |
| | | moderate sample | |
| | | capacity. | |
| Exosome | Altering the | Easy to use, does not | Co-precipitation of other non- |
| Precipitation | solubility or | require specialized | exosomal contaminants like |
| | dispersibility or | equipment, large | proteins and polymeric |
| | exosomes by the | scalable sample | materials. Long run time. |
| | use of water- | capacity | Requires pre- and post cleanup. |
| | excluding | | |
| | polymers | | |
| Immunoaffinity | Exosome fishing | Excellent for the | High reagent cost, exosome |
| capture- | based on specific | isolation of specific | tags need to be established, low |
| based | interaction | exosomes, Highly | capacity and low yields, only |
| techniques | between | purified exosomes - | works with cell-free samples, |
| | membrane- | much better than those | tumor heterogenicity hampers |
| | bound antigens | isolated by other | immune recognition, antigenic |
| | (receptors) of | techniques, high | epitope may be blocked or |
| | exosomes and | possibility of | masked. |
| | immobilized | subtyping. | |
| | antibodies | | |
| | (ligands) | | |
| Microfluidics- | Microscale | Fast, low cost, | Lack of standardization and |
| based | isolation based | portable, easy | large scale tests on clinical |
| techniques | on a variety of | automation and | samples, lack of method |
| | properties of | integration, high | validation, moderate to low |
| | exosomes like | portability. | sample capacity. |
| | immunoaffinity, | | |
| | size, and density | | |

In contrast to past practices, the present subject matter provides, among other things, a multi-step ultracentrifugation process. FIG. 1 shows a typical process flow for the separation, isolation and purification of exosomes during or after a hollow fiber perfusion reactor cell expansion.

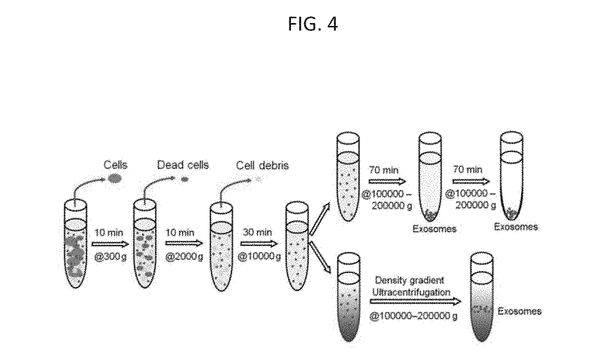

The isolation of exosomes by differential ultracentrifugation typically consists of a series of centrifugation cycles of different centrifugal force and duration to isolate exosomes based on their density and size differences from other components in a sample. For ultracentrifugation, the centrifugal force used typically ranges from ˜100,000 to 120,000 Å˜g. Before the start of isolation, a cleaning step is usually carried out for human plasma/serum to rid of large bioparticles in a sample and the sample is spiked with protease inhibitors to prevent the degradation of exosomal proteins. Between runs during exosome isolation, the supernatant is aspired and depending on the centrifugal force used, either the supernatant or the pellet is re-suspended in an appropriate medium such as phosphate buffered saline (PBS) and subjected to subsequent runs of centrifugation with increasing centrifugal force. Finally, the isolated exosomes are once again re-suspended and stored at −80° C. until further analysis. This method of isolating exosomes is also known as the pelleting method or simple ultracentrifugation method. A schematic illustration of the workflow of differential ultracentrifugation is presented in FIG. 4.

Variations of ultracentrifugation also exist, such as density gradient ultracentrifugation. There are two types of density gradient ultracentrifugation, namely isopycnic ultracentrifugation and moving-zone ultracentrifugation. The use of density gradient ultracentrifugation has become increasingly popular in the isolation of extracellular vesicles like exosomes. In density gradient ultracentrifugation, separation of exosomes is accomplished based on their in size, mass, and density in a pre-constructed density gradient medium in a centrifuge tube with progressively decreased density from bottom to top. A sample is layered as a narrow band onto the top of the density gradient medium and subjected to an extended round of ultracentrifugation. Upon applying a centrifugal force, solutes including exosomes in the sample move as individual zones through the density gradient medium towards the bottom each at its specific sedimentation rate, thus leading to discrete solute zones. The separated exosomes can then be conveniently recovered by simple fraction collection.

While a continuous gradient is used for analytical applications, a discontinuous gradient (stepped gradient) is more suited for preparative purposes in which the separated exosomes are located at the interface of the density gradient layers, thus greatly facilitating their harvesting. Unlike differential ultracentrifugation, a downside of density gradient ultracentrifugation is that the narrow load largely limits its capacity zone. In isopycnic ultracentrifugation, a density gradient medium embracing the entire range of densities of solutes in a sample is loaded to a centrifuge tube. The separation of exosomes from other solutes into a discrete zone exclusively depends on their density difference from those of all other solutes provided that a sufficient period of centrifugation is engaged. During centrifugation, exosomes sediment along the density gradient medium to where they have the same density as the medium—isopycnic position. After the exosomes have reached their isopycnic position, the centrifugal force further focuses the exosomes into a sharp zone and upholds them there, implying that isopycnic ultracentrifugation is static. Alternatively, a sample containing exosomes can be uniformly mixed with a gradient medium in the case of self-generating gradient materials such as cesium chloride. During centrifugation, the exosomes move to their isopycnic position while a density gradient of cesium chloride is generated. Exosomes can then be extricated from the density region of interest between 1.10 and 1.21 g/mL, where they are concentrated. The aliquot obtained from the density region of interest is then subjected to a brief ultracentrifugation at ˜100,000 Å˜g to afford pure exosome pellets which are re-suspended in PBS for further processing and storage.

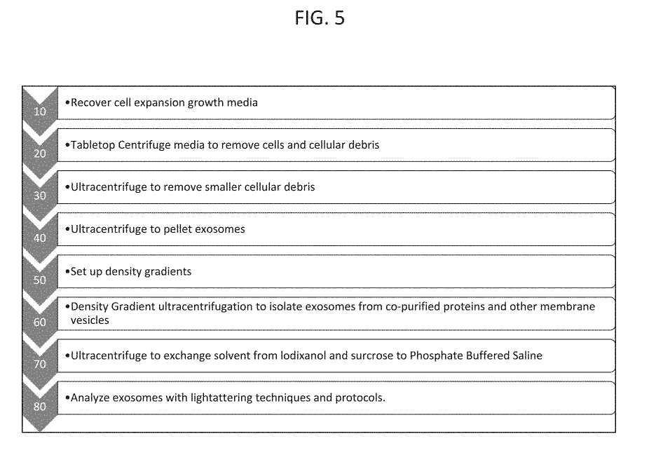

FIG. 5 shows a process flow that can be implemented in commercially available equipment such as a Beckman Coulter Life Sciences, Grants Pass, Oreg. USA, Biomark 4000 automated workstation. The process begins with the media effluent from a hollow fiber cell expansion media exchange which is captured (collected) and used as the source material for the exosome separation. The captured media is centrifuged to sediment any cells in the media, followed by centrifugation to sediment dead cells and larger debris using standard conical tubes. Subsequently the supernatant can be centrifuged to remove cellular debris and filtered prior to exosome pelleting. The exosomes are then pelleted and can then be re-suspended in PBS and layered over a density gradient to isolate the exosomes from any remaining proteins or other membrane vesicles. A semi-final step of exchanging any density gradient solvent from the exosomes can be carried out prior to final optical light scattering analysis. For example, the process may begin with Step 10, where the media effluent from a hollow fiber cell expansion media exchange is captured and used as the source material for the exosome separation. In the next step 20, the captured media is centrifuged for about 10 to 20 minutes at about 500×g to about 1000×g to sediment any cells in the media, followed by about a 10 minute to 20 minute run at about 1500×g to about 2500×g to sediment dead cells and larger debris using standard conical tubes. Subsequently the supernatant can be centrifuged at about 8,000×g to about 12,000×g for about 30 minutes to about 60 minutes at about 4° C. to remove cellular debris and filtered through a 0.2 to about 0.3 micron membrane prior to exosome pelleting. In step 40 the exosomes are pelleted at about 80,000×g to about 120,000×g for about 60 minutes to about 120 minutes. The pellets can then be re-suspended in PBS and layered over a density gradient 50. The density gradient ultracentrifugation can then run for about 12 hours to about 24 hours at about 80,000×g to about 120,000×g at 4° C. to isolate the exosomes from any remaining proteins or other membrane vesicles. A semi-final step of exchanging any density gradient solvent from the exosomes can be carried out at 100,000×g for 1 hour prior to final optical light scattering analysis 70. In one embodiment, the captured media is centrifuged for about up to 20 minutes at up to about 1000×g to sediment any cells in the media, followed by up to a 20 minute run at up to 2500×g to sediment dead cells and larger debris using standard conical tubes. Subsequently the supernatant can be centrifuged at up to about 12,000×g for up to 60 minutes at about 4° C. to remove cellular debris and filtered through a 0.2 to about 0.3 micron membrane prior to exosome pelleting. In step 40 the exosomes are pelleted at up to about 120,000×g for up to about 120 minutes. The pellets can then be re-suspended in PBS and layered over a density gradient 50. The density gradient ultracentrifugation can then run for up to about 24 hours at up to about 120,000×g at 4°.

In another example, the process begins with Step 10, the media effluent from a hollow fiber cell expansion media exchange is captured and used as the source material for the exosome separation, the next step 20 the captured media is centrifuged for 15 minutes at 750 g to sediment any cells in the media, followed by a 15 minute run at 2000×g to sediment dead cells and larger debris using standard conical tubes. Subsequently 30 the supernatant can be centrifuged at 10,000×g for 45 minutes at 4° C. to remove cellular debris and filtered through a 0.22 micron membrane prior to exosome pelleting. In step 40 the exosomes of 30 are pelleted at 100,000×g for 90 minutes using an Optima XPN Ultracentrifuge from Beckman Coulter. The pellets can then be re-suspended in PBS and layered over a density gradient 50. The density gradient Ultracentrifugation can then run for 18 hours at 100,000×g at 4° C. to isolate the exosomes from any remaining proteins or other membrane vesicles. A semi-final step of exchanging any density gradient solvent from the exosomes can be carried out at 100,000×g for 1 hour prior to final optical light scattering analysis 70 The optical analysis can also be implemented with a DelsaMax CORE analysis device or equivalent also supplied by Beckman Coulter. The exosome size and distribution is then recorded and stored with the recovered exosomes.

The above discussion is meant to be illustrative of the principle and various embodiments of the present invention. Although the present invention discloses a method of producing and purifying exosomes, numerous variations and modifications will become apparent to those skilled in the art once the above disclosure is fully appreciated. For example it is possible with a hollow fiber bioreactor to expand all types of human non terminally differentiated cells such as epithelial, hormone-secreting, integumentary system, nervous system, metabolism and storage, extracellular matrix, contractile, blood and immune, germ lines, interstitial and immortal cancer, and harvest exosomes from the growth media effluent for therapeutic and diagnostic uses. It is intended that the following claims be interpreted to embrace all such variations and modifications.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method of purifying exosomes from non-terminally differentiated cells, comprising:
providing a cellular effluent from non-terminally differentiated mammalian cells cultured in media;
separating the cellular effluent into the mammalian cells and a first supernatant comprising exosomes;
separating the first supernatant into cellular debris and a second supernatant comprising exosomes;
filtering the second supernatant into a filtrate comprising exosomes;
subjecting the filtrate to ultracentrifugation to pellet the exosomes;
resuspending the pelleted exosomes and subjecting the resuspended exosomes to density gradient ultracentrifugation to isopycnically isolate the exosomes; and
collecting the isolated exosomes.

2. The method of claim 1 wherein the effluent is a stem cell effluent.

3. The method of claim 1 wherein the cellular effluent, the first supernatant or the second supernatant is separated by centrifugation.

4. The method of claim 3 wherein the cellular effluent is subjected to up to about 1000×g.

5. The method of claim 3 wherein the first supernatant is subjected to up to about 2500×g.

6. The method of claim 3 wherein the second supernatant is subjected to centrifugation before filtration.

7. The method of claim 6 wherein the second supernatant is subjected to up to about 10,000×g before filtration.

8. The method of claim 1 wherein the filtrate is subjected to up to about 200,000×g.

9. The method of any claim 1 wherein the filter is an about 0.2 to about 0.3 micron filter.

10. The method of claim 1 wherein the resuspended exosomes are subjected to up to about 200,000×g for about 18 hours.

11. The method of claim 1 further comprising subjected the isolated exosomes to solvent exchange.

12. The method of claim 1 wherein the effluent is obtained from a hollow fiber bioreactor comprising a plurality of fibers that is part of a sterile closed-loop circuit for media and gas exchange; a gas exchange module; a waste bag; and a cell harvest bag, wherein the fibers are coated with a glycoprotein and a molecule or mixture that promotes cell attachment.

13. The method of claim 1 wherein the mammalian cells are human cells.

14. A method of purifying exosomes from mammalian cells, comprising:
providing an isolated supernatant comprising exosomes and lacking cells, which supernatant is obtained from cultured mammalian cells;
filtering the supernatant to provide a filtrate comprising exosomes;
concentrating the filtrate via ultracentrifugation to provide for isolated exosomes;
subjecting the isolated exosomes to density gradient ultracentrifugation to isopycnically isolate the exosomes; and
collecting the isolated exosomes.

15. The method of claim 14 wherein the cultured cells are non-terminally differentiated cells.

16. The method of claim 14 wherein the filter is an about 0.15 to an about 0.3 micron filter.

17. A method of purifying exosomes from mammalian cells, comprising:
providing an isolated supernatant comprising exosomes and lacking cells, which supernatant is obtained from cultured mammalian cells;
filtering the supernatant to provide a filtrate comprising exosomes;
concentrating the filtrate via ultracentrifugation to provide for isolated exosomes or subjecting filtrate to density gradient ultracentrifugation to isopycnically isolate the exosomes; and
collecting the isolated exosomes.

17. The method of claim 16 wherein the cultured cells are non-terminally differentiated cells.

18. The method of claim 16 wherein the effluent is obtained from a hollow fiber bioreactor comprising a plurality of fibers that is part of a sterile closed-loop circuit for media and gas exchange; a gas exchange module; a waste bag; and a cell harvest bag, wherein the fibers are coated with a glycoprotein and a molecule or mixture that promotes cell attachment.

19. The method of claim 16 wherein the mammalian cells are human cells.

20. The method of claim 16 wherein the cells are stem cells.
